(19) United States
(12) Patent Application Publication
(10) Pub. No.: US 2006/0008849 A1
(43) Pub. Date: Jan. 12, 2006

(54) Methods for measuring chloride channel conductivity

(76) Inventors: Weimin Tang, Princeton, NJ (US); Mary Jo Wildey, Boonton, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/158,001

(22) Filed: Jun. 21, 2005

Related U.S. Application Data
(60) Provisional application No. 60/582,338, filed on Jun. 23, 2004.

(51) Int. Cl. G01N 33/53

(52) U.S. Cl. 435/7.1

(57) ABSTRACT

The invention provides non-radioactive methods to assay for functional chloride channels. The methods colorimetrically detect the amount of iodide conducted by a chloride channel. They can be easily adapted for high throughput assays or screenings.

### CROSS-REFERENCE TO RELATED APPLICATIONS

This application claims priority to Application No. 60/582,338 filed on Jun. 23, 2004.

### FIELD OF THE INVENTION

The invention relates to methods of determining chloride channel conductivity. More particularly, the invention relates to colorimetric detection methods for assaying chloride channel conductivity.

### BACKGROUND OF THE INVENTION

Chloride channels play important physiological roles, including, but not limited to, ion homeostasis, membrane potential regulation, cell volume regulation, transepithelial transport, and regulation of electrical excitability. They are a target class of increasing importance to the pharmaceutical industry, due to their relevance in a wide variety of diseases, such as an impairment of transepithelial transport in cystic fibrosis and Bartter's syndrome, increased muscle excitability in myotonia congenital, reduced endosomal acidification and impaired endocytosis in Dent's disease, and impaired extracellular acidification in osteoclasts and osteopetrosis and blindness. Although different families of chloride ion channels have different structures, they share common functional elements. For example, the channels are all proteinaceous pores in biological membranes that allow the passive diffusion of chloride ion (Cl⁻) along their electrochemical gradient. These channels can also conduct other negatively charged ions such as Br⁻, I⁻, NO₃⁻, HCO₃⁻, SCN⁻, and some small organic acids. They are named chloride channels mainly because chloride is the most abundant anion in biological systems.

There are three well-established gene families of chloride channels: CLC, CFTR, and the ligand-gated GABA and glycine receptors (Jentsch et al., 2002, Physiol Rev. 82: 503-568). Other gene families such as CLIC or CLCA, were also reported to encode chloride channels but are less characterized (Jentsch et al, 2002, supra). The CIC family is the most ubiquitously expressed of all classes of chloride channels established thus far. CLC genes are present both in prokaryotes and eukaryotes. Many CLC channels are voltage-gated, and display a greater conductivity for Cl⁻ than I⁻. It is mutations in the CLC genes that cause diseases such as those mentioned above. CFTR, the cystic fibrosis transmembrane conductance regulator, is a voltage-independent anion channel, which requires the presence of hydrolysable nucleoside triphosphates for efficient activity. CFTR has greater anion permeability for Cl⁻ than I⁻ and is associated with cystic fibrosis. GABA and glycine receptors are ligand-gated chloride channels. GABA (γ-aminobutyric acid) and glycine are the primary neurotransmitters for the fast inhibitory neurotransmission in mammalian central nervous system (CNS). They bind to their receptors and open intrinsic anion channels, leading to a Cl⁻ influx or efflux depending on the electrochemical driving force. Both GABA and glycine receptors show a greater permeability for I⁻ than Cl⁻. They are targets for a wide range of clinically important drugs, including antiepileptic agents, anxiolytics, sedatives, hypnotics, muscle relaxants, and anesthetics.

While identifying new drugs to modulate chloride channel activity is needed, there are not readily available high affinity ligands for chloride channels. In contrast, cation channels have highly specific channel blockers that are often derived from animal toxins making the development of screening assays more straight forward. Chloride channel blockers are rather unspecific and have a low potency with effective blocking concentrations in the range of micromolar to even millimolar.

The existing technologies for identifying a modulator for a chloride channel are a compromise between throughput, physiological relevance, sensitivity and robustness. The best-known assay today is probably the patch-clamp technique. The patch-clamp technique controls the electrical potential difference across a small patch of membrane or across the plasma membrane of an entire cell. The technique directly assesses the current carried by ions crossing the membrane at that voltage through ionic channels. This technology provides high quality and physiologically relevant data of ion-channel function at the single cell or single channel (within a small patch of membrane) level. But, setting up patch-clamping experiments is a complicated process requiring highly trained personnel to make the system less vulnerable to interference from vibration and electrical noise. Throughput of a veteran patch-clamper is, at best, 10-30 data points per day (Xu, et al. (2001), Drug Discovery Today, 6:1278-12887). Such low throughput and high labor-cost is far from acceptable for high throughput screen (HTS) purposes. Although several companies are attempting to automate the patch-clamp process, the current complexity and reproducibility of the experimental setup renders it unsuitable for an HTS application.

Technologies based on flux assays are currently available in a fully automated high throughput format for ionic channel drug screening. Flux assays have been used for functional studies of chloride channels (See review, Sikander et al., Assay and Drug Development Technologies (2003), 1 (5), 709-717). Because the intracellular chloride ion (Cl⁻) concentration is high, it is difficult to detect changes in choride channel conductivity by calorimetrically measuring changes in the submicromolar concentration of chloride ions. Thus, radiolabeled ³⁶Cl⁻ or ¹²⁵I⁻ ionic influx has been used to measure chloride channel conductivity. Radiolabeled ³⁶Cl⁻ or ¹²⁵I⁻, or Cl⁻-sensitive fluorescent indicators, has also been used to measure the ionic efflux from a chloride channel.

A sensitive, non-radioactive, quantitative assay method for chloride channels that is easily adaptable to high-throughput screening (HTS) format is needed.

### SUMMARY OF THE INVENTION

This invention provides calorimetric methods to assay for functional chloride channels. The methods can be easily adapted for high throughput assays or screenings.

Other aspects, features and advantages of the invention will be apparent from the following disclosure, including the detailed description of the invention and its preferred embodiments and the appended claims.

### BRIEF DESCRIPTION OF THE DRAWINGS

FIG. 1 illustrates a concentration titration curve of standard samples of NaI tested with the Sandell-Kolthoff (SK) assay. The standard samples comprised various concentrations of NaI as provided on the horizontal axis. Each data point represents the average of 8 samples having the same NaI concentration. The OD₄₀₅ absorbance was measured after the reaction was incubated at room temperature for 15 minutes (FIG. 1A) or 10 (filled square), 15 (upward triangle), 20 (downward triangle), 25 (diamond), 30 (circle), and 70 minutes (open square) (FIG. 1B).

FIG. 2 illustrates that the conductivity, expressed as activity %, of outwardly rectifying GABAA channel increased with increasing amount of GABA as measured by the SK assay.

FIG. 3 shows that stimulating cells with 30 μM GABA results in approximately a 4-fold decrease in OD₄₀₅ in the SK assay, thus a 4-fold increase in iodide concentration, as compared to cells not stimulated with GABA. Each data point represents the average of 4 samples stimulated with the same GABA concentration.

FIG. 4 demonstrates that the conductivity of outwardly rectifying GABAA channel decreased with increasing amount of non-competitive inhibitor (Picrotoxin, triangle) or competitive blocker (Bicuculline, square) for GABAA channel as measured by the SK assay. FIG. 4A. In the presence of 30 μM of GABA; and FIG. 4B. In the presence of 300 μM of GABA.

FIG. 5 illustrates that the conductivity of outwardly rectifying CFTR chloride channel increased with increasing amount of Forskolin, an activator for the channel, as measured by the SK assay. FIG. 5A. The assay was performed with cells having endogenous functional CFTR channel; FIG. 5B. The assay was performed with cells having a defective CFTR channel.

FIG. 6 demonstrates that the inwardly rectifying GABAA channel conductivity (expressed as activity %) increased with increasing amount of GABA as measured by the SK assay.

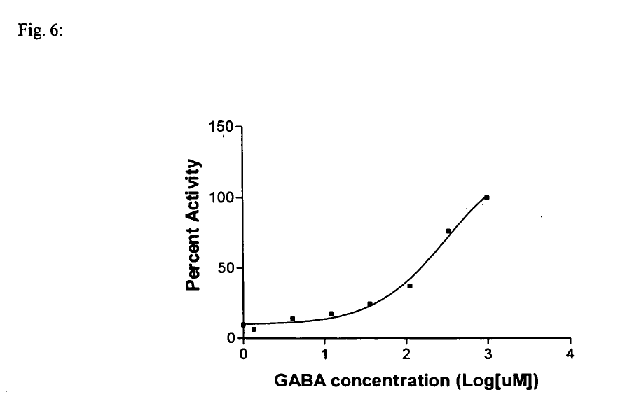

### DETAILED DESCRIPTION OF THE INVENTION AND ITS PREFERRED EMBODIMENTS

All publications cited below are hereby incorporated by reference. Unless defined otherwise, all technical and scientific terms used herein have the same meaning as commonly understood to one of ordinary skill in the art to which this invention pertains.

As used herein, the terms “comprising”, “containing”, “including”, and “having” are used in their open, non-limiting sense.

This invention provides colorimetric detection methods to study chloride channel conductivity using non-radioactive iodide as the tracer. Most chloride channels conduct iodide and the intracellular concentration of iodide is very low. Thus, a method for measuring the conductivity of a chloride channel according to this invention comprises the steps of: a) contacting a chloride channel with iodide; and b) calorimetrically detecting the amount of iodide conducted by the chloride channel.

As used herein, “a colorimetric detection method” refers to a method comprising the step of detecting a colored agent in a test sample as an indicator of the iodine concentration in that sample. Reliable and sensitive “colorimetric detection methods” have been used in surveys of iodine content in food or biological samples such as urine samples (See for example, Yaping Z. et al., Clin Chem. 1996 December; 42 (12):2021-7). Methods of the present invention can utilize a variety of “calorimetric detection methods” that have been used or are yet to be developed to determine the amount of iodine in a test sample. Often, such calorimetric detection methods are based on the catalytic effect of iodine (I) or its ions such as iodide (I⁻) or iodate (IO₃⁻).

In one preferred embodiment, the “colorimetric detection method” that can be used to determine the amount of iodide in a test sample is derived from the method of Sandell and Kolthoff (SK method) (Sandell et al., 1937, Mikrochem. Acta, 1:9). The SK method is based on the Sandell and Kolthoff reaction: 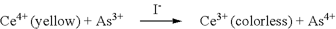
The SK method makes use of the catalytic effect of iodide (I⁻) on the reduction of the yellow colored cerium ion (Ce⁴⁺) to colorless Ce³⁺ by arsenious acids. The more iodide that is in a test sample, the faster the SK reaction is, and the quicker the yellow colored Ce⁴⁺ disappears. The amount of Ce⁴⁺ in the test sample can be determined by the absorbance of light, which is defined as the amount of light that is absorbed by the liquid comprising the test sample. In particular, the absorbance can be measured using a colorimeter or a spectrophotometer, by beaming light at a given wavelength through the liquid sample, and measuring the amount of light that goes through the liquid sample. For example, the amount of Ce⁴⁺ in the test sample can be measured by the absorbance of light at a wavelength of about 405 nm (OD₄₀₅). Examples of colorimetric detection methods based on the SK reaction are provided infra for the purpose of demonstration. The SK detection system is sensitive and reliable, and has been suggested as the global standard iodine detection method by the World Health Organization (WHO).

In another embodiment, the “colorimetric detection method” that can be used to determine the amount of iodide in a test sample is an iodide conversion test. In this test iodide is first converted into iodine and then the amount of iodine is determined using a starch-iodine test (Wade, 1925, Ind. Eng. Chem., 17: 470). Iodide can be oxidized into free iodine by reacting it with any suitable oxidant, such as chlorine. Free iodine is capable of forming a blue colored complex with starch. The more iodide that is in a reaction mixture, the more blue colored starch-iodine complex will be formed. The amount of the starch-iodine complex in the reaction mixture can be determined by the absorbance of light of the aqueous (upper) phase, for example, at a wavelength of 480 nm (OD₄₈₀) (Kozutsumi et al., 2000, Cancer Letters, 158:93-98).

Another example of “calorimetric detection methods” that can be used to determine the amount of iodide in a test sample is derived from the method of Sveikina (Moxon et al., 1980, Analyst, 105:344-352; and Kenneth O. et al., 2001, Polish Journal of Food and Nutrition Sciences, 10: 35-38). In this method, iodide catalyses the destruction of thiocyanate by nitrite, with an accompanying decrease in the orange color of the iron (III) thiocyanate produced by the addition of iron (III) ions. The more iodide that is in the sample, the less the iron (III) thiocyanate will be produced. The amount of iron (III) thiocyanate can be determined by the absorbance of light, for example at a wavelength of about 430 nm.

Yet another example of “calorimetric detection methods” that can be used to determine the amount of iodide in a test sample is based on the iodide-catalyzed oxidation of 3,3′,5,5′-tetramethylbenzidine (TMB) by peracetic acid/H2O2, to yield colored products (Rendl et al., 1998, J. Clin Endocrinol Metab, 83 (3):1007-12). The first colored product is a blue charge-transfer complex of the parent diamine and the diamine oxidation product. This species exists in rapid equilibrium with the TMB-radical cation. With high iodide concentrations, the test sample turns blue, passes through a green stage, and finally becomes yellow. The amount of iodide in a test sample can be quantitatively measured by the absorbance of light, for example at a wavelength of about 655 nm.

The colorimetric detection method can be utilized in connection with any chloride channel. Such chloride channels include, but are not limited to, a voltage-gated chloride channel, a ligand-gated chloride channel, a swelling-activated chloride channel, a calcium-activated chloride channel, and a CLIC chloride channel. Preferred chloride channels that can be assayed using the method of the invention are CLC, CFTR, and the ligand-gated GABA and glycine receptors.

Because chloride channels allow passive diffusion of anions, their activation can lead to a passive influx or efflux of anions, depending on the electrochemical potential for the anion. As used herein, the “influx” of anions into a system through a chloride channel refers to the process of anions outside the system coming into the system via the chloride channel integrated on the surface of the system. The “influx” of anions into a cell or membrane vesicle refers to the process of anions outside the cell or membrane vesicle coming into the cell or membrane vesicle via a chloride channel situating in the cell membrane or the membrane of the membrane vesicle. As used herein, the “efflux” of anions from a system through a chloride channel refers to the process of anions inside the system coming out of the system via the chloride channel integrated on the surface of the system. The “efflux” of anions out of a cell or membrane vesicle refers to the process of anions inside the cell or membrane vesicle coming out of the cell or membrane vesicle via a chloride channel situated in the cell membrane or the membrane of the membrane vesicle. For example, upon activation, a CFTR or a GABA receptor can mediate efflux of anions out of the cell, and a GABA receptor can also mediate influx of anions into the cell.

The methods of this invention can be used to measure the influx of iodide into a system comprising a chloride channel, as well as the efflux of iodide out of a system comprising a chloride channel. As used herein, a “system comprising a chloride channel” refers to any structurally discrete component having a phospholipid bilayer membrane on the surface of the component, and a chloride channel integrated in the membrane.

In one preferred embodiment, the “system comprising a chloride channel” can be a cell expressing the chloride channel. The cell can be a microbial cell, such as a bacterial cell or a yeast cell, a plant cell, or an animal cell, such as a cell derived from a human, mouse, rat, or other mammals. The cell can be a natural host cell that expresses the chloride channel of interest endogenously. For example, many epithelia cells can be natural hosts for CFTR channel, and neurons can be the natural hosts for GABA receptor. Preferable, the chloride channel of interest is the only or predominant type of chloride channel that is active under the assay condition. Besides using a method described infra to specifically activate the channel of interest, methods are known to those skilled in the art to inactivate undesired chloride channels in the cell. For example, the undesired chloride channel in the cell can be inactivated temporarily by subjecting the cell to a specific chemical, such as a blocker or inhibitor for the channel. Or, the undesired chloride channel can be inactivated permanently by genetic manipulation such as gene knock out or anti-sense technology.

The cell expressing the chloride channel can also be a recombinant host cell. Cells can be transfected with a nucleic acid molecule that is capable of expressing a chloride channel of interest. The chloride channel gene can be expressed, for example, from a vector that is either stably or transiently transfected into the cell. Vectors suitable for gene expression are known in the art and many are commercially available.

In another preferred embodiment, the “system comprising a chloride channel” can be a membrane vesicle comprising a chloride channel in the membrane. The membrane vesicles can be prepared from the biological membranes, such as the tissue membrane, plasma membrane, cell membrane, or internal organelle membrane comprising the chloride channel. For example, CFTR is expressed in the apical membrane of various eithelia, most prominently in those of the intestine, airways, secretory glands, bile ducts, and epididymis. Membrane vesicles of such apical membrane can be used to study CFTR. Methods are known to those skilled in the art for isolation and preparation of biological membrane vesicles. For example, such a method can include the steps of mechanical or enzymatic disruption of the tissue or cells, centrifugation to separate the membranes from other component, and resuspending the membrane vesicles in suitable buffer solution.

The membrane vesicle can also be prepared from artificial membranes. Purified chloride channel protein can be reconstituted into lipid bilayers to form the artificial membrane vesicles (see Chen et al., 1996, J. Gen. Physiol. 108:237-250). These membrane vesicles can contain few proteins and can be manufactured to contain at least one and perhaps only one type of chloride channel protein thereby focusing the data to reflect vescicles containing a single type of chloride channel. Methods for the preparation of artificial membrane vesicles are known to those in the art.

The membrane vesicle can further be a subcellular organelle with a chloride channel present in the membrane of the organelle. Examples of subcellular organelles that can be used in the present methods include, but are not limited to, mitochondria, golgi apparatus, lysosomes, and endosomes. Methods are known to those skilled in the art to isolate or enrich subcellular organelles.

In some embodiments, membrane vesicles comprising the chloride channels of interest can provide an easier format, because cell lysis and/or shear is not as much of a concern during the assay. In other embodiments, however, cells expressing the chloride channels of interest are preferred, for example, when the cell membrane preparation procedure destroys or inactivates the channel of interest.

In one embodiment, the conductivity of a chloride channel is measured by the amount of iodide influx into a cell or membrane vesicle having the chloride channel. Such a method comprises the steps of: a) incubating a cell or membrane vesicle having the chloride channel in a liquid solution comprising iodine, separating the cell or membrane vesicle from the liquid solution; and b) measuring the amount of iodide inside the cell or membrane vesicle using a calorimetric detection method. The contents inside the cell or membrane vesicle can be released or extracted by lyses or physical disruption. The amount of iodide inside the contents can be measured by any of calorimetric detection methods described supra. The more iodide that is found inside the cell or membrane vesicle, the stronger conductivity of the chloride channel to anions.

In another embodiment, the conductivity of a chloride channel is measured by the amount of iodide efflux out of an iodide-loaded cell or membrane vesicle having the chloride channel. Such a method comprises the steps of: a) incubating the iodide-loaded cell or membrane vesicle having the chloride channel in a liquid solution that is substantially free of iodine; b) separating the cell or membrane vesicle from the liquid solution; and c) measuring the amount of iodide in the liquid solution using a calorimetric detection method.

An “iodide-loaded cell or membrane vesicle” is a cell or membrane vesicle that has been incubated with a liquid solution comprising iodide prior to the step (a) of the method. In one embodiment, the “iodide-loaded cell or membrane vesicle” is washed with an iodide-free liquid solution or a liquid solution that is substantially free of iodine after it has been incubated with iodide. Example 3 infra illustrates a method on how to prepare an “iodide-loaded cell or membrane vesicle”.

A “liquid solution that is substantially free of iodine” refers to a liquid solution that contains no or a very minor amount of iodine or ions thereof, such as iodide or iodide. For example, a “liquid solution that is substantially free of iodine” can have less than about 1 nM iodine or ions thereof. The more iodide is found in the solution, the stronger conductivity of the chloride channel to anions.

In some embodiments, the method to measure the iodide efflux comprises the step of measuring the amount of iodide inside the cell only. The lower the iodide concentration within the cell, the stronger conductivity of the chloride channel to anions.

In other embodiments, the method to measure the iodide efflux further comprises the step of determining the ratio of the amount of iodide in the solution to the amount of iodide inside the cell. The ratio can be used as an indicator for the function of the chloride channel. The higher the ratio, the stronger conductivity of the chloride channel to anions.

The methods of the invention can further comprise the step of activating or opening the chloride channel of interest prior to the measurement of iodide concentration. As used herein, “activating or opening” a chloride channel includes means that results in increased ion conduction by the chloride channel. Depending on the type of the channel, a chloride channel can be activated or opened by different means. Some chloride channels, such as many CLC channels, are voltage-gated. Therefore, electrical signals, such as electrical pulses can be used to regulate (open/close) the conductivity of CLC channels. Some chloride channels are regulated by ligands, therefore can be activated upon addition of small molecules. For example, CFTR requires the presence of cAMP for efficient activity; native Ca²⁻-activated Cl⁻ channels require the presence of intracellular Ca²⁺ for activation; the GABA receptor requires GABA for activation; the glycine receptor requires glycine for activation, etc. In addition, some chloride channels can be activated by cell swelling, i.e., the increase of cell volume.

One general aspect of the invention is that methods of the invention can be used to analyze cells or membrane preparations for the presence of functional chloride channels. Particularly, methods of the inventions can be used to evaluate the proper function of chloride channel in a patient by analyzing cells or membrane preparations derived from clinical samples taken from the patient.

Malfunction of chloride channels has been implicated in many disease states. For example, mutations in CFTR prevent normal passage of chloride ions through the cell membrane (Welsh et al., Neuron, 8:821-829 (1992)). This results in reduced chloride ion permeability in the secretory and absorptive cells of organs with epithelial cell linings, including the airways, pancreas, intestine, sweat glands and the male genital tract. This, in turn, reduces the transport of water across the epithelia, and causes cystic fibrosis. The lungs and the GI tract are the predominant organ systems affected in this disease and the pathology is characterized by blocking of the respiratory and GI tracts with viscous mucus. Methods of the inventions can be used as one of the tests to diagnose whether a patient suffers from cystic fibrosis or not.

Another general aspect of the invention is that methods of the invention can be used to determine an effect of a test compound on the conductivity of a chloride channel. Such a method comprises the steps of: a) contacting the chloride channel with the test compound and iodide; b) colorimetrically detecting the amount of iodide conducted by the chloride channel; and c) comparing the amount of iodide detected with that of a control wherein the chloride channel is not contacted with the test compound. The amount of incubation time required for the contacting steps can be empirically determined, for example, by running a time course with a known chloride channel modulator, and measuring cellular changes as a function of time.

In one embodiment, the method measures the influx of iodide into a cell or membrane vesicle having the chloride channel, comprising the steps of: incubating the cell or membrane vesicle having the chloride channel in a liquid solution containing iodide; contacting the cell or membrane vesicle with the test compound; separating the cell or membrane vesicle from the liquid solution; measuring the amount of iodide inside the cell or membrane vesicle using a calorimetric detection method; and comparing the amount of iodide measured with that of a control, wherein the chloride channel is not contacted with the test compound. A test compound that increases the influx of anion into a system through a chloride channel will result in higher concentrations of iodide inside the system as compared to that of the control. A test compound that decreases (or increases) the influx of anions into a cell or membrane vesicle through a chloride channel will result in lower (or higher) amount of iodide inside the cell or membrane vesicle as compared to that of the control.

In another embodiment, the method measures the efflux of iodide out of an iodide-loaded cell or membrane vesicle having the chloride channel, comprising the steps of: incubating the iodide-loaded cell or membrane vesicle having the chloride channel in a liquid solution that is substantially free of iodine; contacting the cell or membrane vesicle with the test compound; separating the cell or membrane vesicle from the liquid solution; measuring the amount of iodide in the liquid solution using a colorimetric detection method; and comparing the amount of iodide measured with that of a control where the chloride channel is not contacted with the test compound. A test compound that decreases (or increases) the efflux of anions into a cell or membrane vesicle through a chloride channel will result in lower (or higher) amount of iodide in the liquid solution as compared to that of the control.

In some embodiments, the method to measure the iodide efflux comprises the step of measuring the amount of iodide inside the cell only. The lower the iodide concentration within the cell, the stronger conductivity of the chloride channel to anions.

In other embodiments, the method to measure the iodide efflux further comprises the step of determining the ratio of the amount of iodide in the solution to the amount of iodide inside the cell. The ratio can be used as an indicator for the function of the chloride channel. A test compound that decreases (or increases) the efflux of anions into a cell or membrane vesicle through a chloride channel will result in lower (or higher) such a ratio as compared to that of the control.

The compound identification methods described herein can be performed using conventional laboratory formats or in assays adapted for high throughput. The term “high throughput” refers to an assay design that allows easy screening of multiple samples simultaneously, and can include the capacity for robotic manipulation. Another desired feature of high throughput assays is an assay design that is optimized to reduce reagent usage, or minimize the number of manipulations in order to achieve the analysis desired. Examples of assay formats include 96-well or 384-well plates, levitating droplets, and “lab on a chip” microchannel chips used for liquid handling experiments. It is well known by those in the art that as miniaturization of plastic molds and liquid handling devices are advanced, or as improved assay devices are designed, that greater numbers of samples can be performed using the design of the present invention.

Test compounds or candidate compounds encompass numerous chemical classes, although typically they are organic compounds. Preferably, they are small organic compounds, i.e., those having a molecular weight of more than 50 yet less than about 2500. Candidate compounds comprise functional chemical groups necessary for structural interactions with polypeptides, and typically include at least an amine, carbonyl, hydroxyl or carboxyl group, preferably at least two of the functional chemical groups and more preferably at least three of the functional chemical groups. The candidate compounds can comprise cyclic carbon or heterocyclic structure and/or aromatic or polyaromatic structures substituted with one or more of the above-identified functional groups. Candidate compounds also can be biomolecules such as peptides, saccharides, fatty acids, sterols, isoprenoids, purines, pyrimidines, derivatives or structural analogs of the above, or combinations thereof and the like. Where the compound is a nucleic acid, the compound typically is a DNA or RNA molecule, although modified nucleic acids having non-natural bonds or subunits are also contemplated.

Candidate compounds are obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random peptides, and the like. Candidate compounds can also be obtained using any of the numerous approaches in combinatorial library methods known in the art, including: biological libraries; spatially addressable parallel solid phase or solution phase libraries: synthetic library methods requiring deconvolution; the “one-bead one-compound” library method; and synthetic library methods using affinity chromatography selection (Lam (1997) Anticancer Drug Des. 12:145). Alternatively, libraries of natural compounds in the form of bacterial, fungal, plant and animal extracts are available or readily produced. Additionally, natural and synthetically produced libraries and compounds can be readily modified through conventional chemical, physical, and biochemical means.

Further, known pharmacological agents can be subjected to directed or random chemical modifications such as acylation, alkylation, esterification, amidation, etc. to produce structural analogs of the agents. Candidate compounds can be selected randomly or can be based on existing compounds that bind to and/or modulate the function of chloride channel activity. Therefore, a source of candidate agents is libraries of molecules based on a known compound that increases or decreases the conductivity of a chloride channel, in which the structure of the known compound is changed at one or more positions of the molecule to contain more or fewer chemical moieties or different chemical moieties. The structural changes made to the molecules in creating the libraries of analog activators/inhibitors can be directed, random, or a combination of both directed and random substitutions and/or additions. One of ordinary skill in the art in the preparation of combinatorial libraries can readily prepare such libraries.

A variety of other reagents also can be included in the method. These include reagents such as salts, buffers, neutral proteins (e.g., albumin), detergents, etc. that can be used to facilitate optimal protein-protein and/or protein-nucleic acid binding. Such a reagent can also reduce non-specific or background interactions of the reaction components. Other reagents that improve the efficiency of the assay such as nuclease inhibitors, antimicrobial agents, and the like can also be used.

Examples of methods for the synthesis of molecular libraries can be found in the art, for example in: Zuckermann et al. (1994). J. Med. Chem. 37:2678. Libraries of compounds can be presented in solution (e.g., Houghten (1992) Biotechniques 13:412-421), or on beads (Lam (1991) Nature 354:82-84), chips (Fodor (1993) Nature 364:555-556), bacteria (U.S. Pat. No. 5,223,409), spores (U.S. Pat. No. 5,571,698), plasmids (Cull et al. (1992) Proc. Natl. Acad. Sci. USA 89:1865-1869) or phage (see e.g., Scott and Smith (1990) Science 249:3 86-390).

The present invention provides colorimetric detection methods for functional analysis of chloride channels. Preferred embodiments of the invention provide a number of advantages compared to other related methods. For example, there is no radioactive material involved in method of the invention, resulting in reduced cost in terms of resources and reagents required and reduced waste. In addition, methods of the invention are sensitive, can detect iodine concentration as low as 0.01 PPM. Further, methods of the invention can be easily adapted into a high throughput format.

To further illustrate the invention, the following examples are provided.

### EXAMPLE 1

### The Sandell-Kolthoff (SK) Assay on The Standard NaI Solutions

Materials

All chemicals were purchased from Sigma Aldrich Corp. (St Louis, Mo.) except otherwise indicated.

The following procedure was used to prepare the arsenic acid mixture: 1) 19.8 g of arsenic trioxide (As₂O₃) was dissolved in a solution consisting of 300 ml of purified water and 50 ml of ammonia hydroxide (25%); 2) 32 ml of sulfuric acid and 25 g of ammonium chloride (NH₄Cl) were added to the solution; and 3) purified water was added to bring the final volume of the solution to 1000 ml.

The following procedure was used to prepare the ammonia-Ce(IV)-sulfate mixture: 1) 10 g of ammonia-Ce(IV)-sulfate ((NH₄)₄Ce(SO₄)₄.2H₂O) was suspended in 400 ml purified water; 2) 26 ml of sulfuric acid was added to the solution to help dissolve the ammonia-Ce(IV)-sulfate; and 3) after the yellow salt was dissolved, purified water was added to bring the final volume of the solution to about 500 ml.

The standard NaI solutions were prepared by first dissolving NaI in purified water to a final concentration of 100 PPM, then making a 1:10 serial dilution of the 100 PPM solution in 96-well plates (Cat #3903, Corning) to final concentrations of about 10, 1, 0.1, 0.01, and 0.001, 0.0001, and 0.00001 PPM.

Procedures

The following reagents were mixed in a well of a 96-well plate: 100 μl of NaI standard solution, 100 μl of arsenic acid mixture, and 100 μl of ammonia-Ce(IV)-sulfate mixture. The reaction mixture was incubated at room temperature for about 30 minutes. Because iodide catalyzes the reduction of the yellow colored cerium ion (Ce⁴⁺) in ammonia-Ce(IV)-sulfate by arsenic acid to colorless Ce³⁺, the more iodide in the reaction mixture, the less ammonia-Ce(IV)-sulfate would remain in the mixture. The amount of ammonia-Ce(IV)-sulfate in the reaction mixture was measured as OD405 of the reaction mixture using a spectrometer (Spectrometer Plus, Molecular Device, CA).

Results

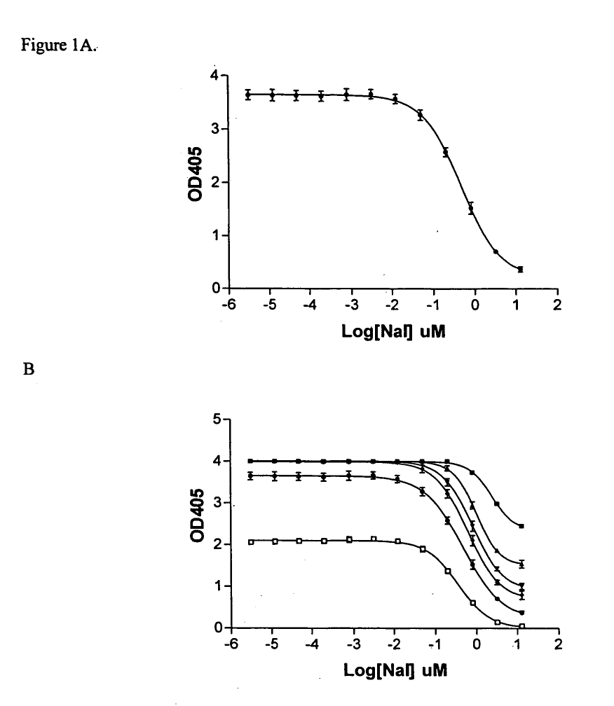

As shown in FIG. 1, while the amount of NaI increased from 0.01 to 10 PPM, decreasing value of OD405 was measured from the SK assay because increasing amount of ammonia-Ce(IV)-sulfate in the reaction mixture was converted to colorless Ce³⁺. Using the SK assay described herein, as low as 0.01 PPM of r could be detected. The signal change is about linear within the range of 0.01 to 10 PPM

### EXAMPLE 2

### The Sandell-Kolthoff (SK) Assay on The Outwardly Rectifying GABAA Receptor

Materials

Similar chemicals and reagents as those described in Example 1 were used in this Example. In addition, the iodine loading buffer consisting of 150 mM NaI, 2 mM CaCl₂, 0.8 mM NaH₂PO₄, 1 mM of MgCl₂, and 5 mM of IK, 2% FBS (# 35-010-AV, CELLGRO, VA) pH7.4, was prepared by mixing and dissolving each described component into purified water, and adjusting the pH accordingly.

Cell line expressing human GABAA (Adenovirus type) was obtained from the American Type Culture Collection (ATCC, Cat No. CRL-2029). Cells were grown in supplemented DMEM medium consisting of DMEM medium (#10-017-CV, CELLGRO, VA), 4 mM L-glutamine, 1.5 g/L sodium bicarbonate, 4.5 g/L glucose, 1.0 mM sodium pyruvate, and 10% fetal bovine serum (# 35-010-AV, CELLGRO, VA)

Procedure

Cells in supplemented DMEM medium (200 μl, approx. 250,000 cells/ml) were added to each well of a D-lysine coated 96-well plate (Corning, Cat No. 3667), and were incubated overnight in a tissue culture incubator at 37° C. under 90% air/10% CO₂. Then, the supplemented DMEM medium was removed with a multichannel pipettor, and 200 μl iodine-loading buffer was added to each well with Rapid Plate™ (Zymark, MA). Cells were incubated for 2-4 hours at 37° C. under 90% air/10% CO₂, and were subsequently washed with phosphate buffer saline (DPBS, Invitrogen, CA), or culture medium. DPBS (100-200 μl) was added to each well. GABA was added to each well at a final concentration of 100, 30, 10, 3, 1, 0.3, 0.1, or 0 μM with Zymark Rapid plate (Zymark, MA). In some assays, in addition to the GABA, a test compound such as the known GABAA channel antagonist, Picrotoxinin (P-8390, Sigma, MO, Khrestchatisky et al., 1989, Neuron 3:745-53) or Bicuculline (B-6889, Sigma), was also added to the cells. After the cells were incubated with GABA in the presence or absence of the test compound for 5 minutes, they were separated from the suspending buffer, and were lysed with 100 μl of cell lysis buffer (1% Triton X-100). The amount of I⁻ in the lysed cells was measured by the SK assay procedure described in Example 1.

Results

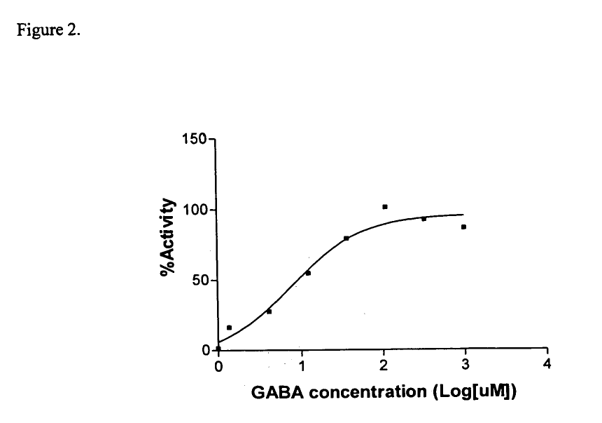

FIG. 2 showed that the outward conductivity of GABAA channel increases with increasing amount of GABA as measured by the SK assay. GABA activated the GABAA channel resulting in the efflux of iodide from the cell. As shown in FIG. 1, the more iodide in the reaction mixture, the less the absorbance value at OD₄₀₅ would be measured from the SK assay. The conductivity of GABAA channel is expressed as the percent activity of the channel, which is defined as: 100*(OD₄₀₅ₛₐₘₚₗₑ−OD_{405low})/(OD_{405high}−OD_{405low}), wherein OD₄₀₅ₛₐₘₚₗₑ is measured from the SK assay on cells treated with various concentrations of GABA; OD_{405high} is measured from the SK assay on cells treated with 300 μM of GABA; and OD_{405low} is measured from the SK assay on cells without GABA treatment. The measured EC₅₀ of GABA, which is the concentration of GABA at which the activity of the GABAA channel is induced by one-half as compared to reactions with 300 μM of GABA, was 7.69+/−0.3 μM.

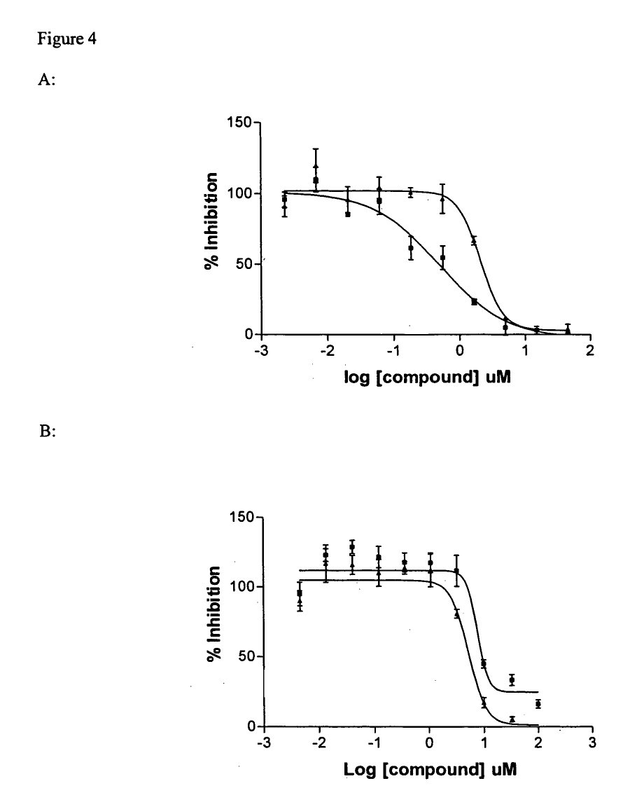

FIG. 4 showed that the conductivity of the GABAA channel decreased with increasing amount of non-competitive inhibitor or competitive blocker for GABAA channel as measured by the SK assay. Again, the conductivity of the GABA channel is expressed as the percent activity of the channel as defined supra. Under the assay condition described herein, the GABAA channel non-competitive inhibitor Picrotoxin had an IC₅₀ of about 5.3 μM in the presence of 30 μM GABA, and an IC₅₀ of about 10 μM in the presence of 300 μM of GABA. The GABA channel competitive blocker Bicuculline had an IC₅₀ of about 1 μM in the presence of 30 μM of GABA and an IC₅₀ of about 50 μM in the presence of 300 μM of GABA. The IC₅₀ of a test compound in the presence of a given GABA concentration is the concentration of the test compound at which the conductivity of the GABAA channel is decreased by one-half as compared to reactions without the test compound but with the same concentration of GABA. IC₅₀s were calculated with IDBS XL-fit model 205 (IDBS, UK).

Similar assay procedures as the one described in this example can be used to measure other types of outwardly rectifying ligand-gated chloride channels.

### EXAMPLE 3

### The Sandell-Kolthoff (SK) Assay on The Outwardly Rectifying CFTR channel

Materials

Similar chemicals and reagents as those described in Example 2 were used in this Example.

HTB-79 cell line intrinsically expressing the human CFTR channel was obtained from ATCC. The CRL-1918 cell line, having a defective CFTR channel, was also obtained from ATCC. Cells were grown in Iscove's modified Dulbecco's medium consisting of Iscove's modified medium (CELLGRO, VA) and 4 mM L-glutamine, 1.5 g/L sodium bicarbonate, and 20% FBS (CELLGRO, VA).

Procedure

HTB-79 cells in Iscove's modified Dulbecco's medium (200 μl, approx. 500,000 cells/ml) were added to each well of a costar 96-well plate (Corning Costar, NY), and were incubated overnight in a tissue culture incubator at 37° C. under 90% air/5% CO₂. Then, the Iscove's modified Dulbecco's medium was removed and 200 μl of iodine-loading buffer was added to each well of the plate. Cells were incubated for 2-4 hours at 37° C. under 90% air/5% CO₂ and washed with DPBS (Invitrogen, CA) or culture medium. DPBS (100-200 μl) was added to each well. Forskolin (Sigma, MO) was added to each well at a final concentration of 100, 30, 10, 3, 1, 0.3, 0.1, 0.03 and 0.01 μM. After the cells were incubated at room temperature for an additional 5 minutes, they were separated from the suspending buffer, and were lysed with 100 μl of cell lysis buffer (1% Triton X-100). The amount of I⁻ in the lysed cells was measured by the SK assay procedure described in Example 1.

Results

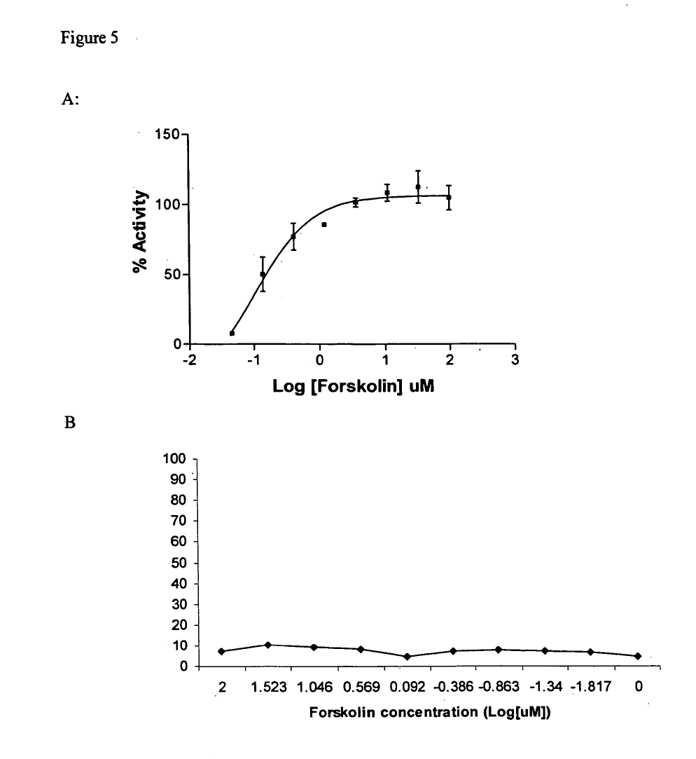

FIG. 5 showed that as measured by the SK assay, increasing amount of Forskolin caused increasing conductivity of the CFTR channel. Forskolin stimulated adenylate cyclase activity resulting in increased level of cAMP, which in turn activated CFTR channel. FIG. 5A showed that as measured by the SK assay, Forskolin activated chloride channel conductivity in HTB-79 cells, which endougeneously express CFTR channels. The measured EC₅₀ for Forskolin was 1 μM. The EC₅₀ for Forskolin is the concentration of Forskolin at which the activity of the CFTR channel is induced by one-half as compared to reactions with 100 μM Forkolin. The SK assay could detect the activation of CFTR by Forskolin at a concentration as low as 300 nM. FIG. 5B showed that as measured by the SK assay, up to a concentration of 100 μM, Forkolin did not activate chloride channel conductivity in CRL-1918 cells, which express a defective CFTR channel. The conductivity of the CFTR receptor is expressed as the percent activity of the channel, which is defined as: 100*(OD₄₀₅ₛₐₘₚₗₑ−OD_{405low})/(OD_{405high}−OD_{405low}), wherein OD405ₛₐₘₚₗₑ is measured from the SK assay on cells treated with Forskolin; OD405_{low} is measured from the SK assay on cells treated with 100 μM of Forkolin; and OD405_{high} is measured from the SK assay on cells without Forskolin treatment.

### EXAMPLE 4

### The Sandell-Kolthoff (SK) Assay on the Inwardly Rectifying GABAA Receptor

Materials

Similar chemicals and reagents as those described in Example 2 were used in this example.

Procedure

Cells in supplemented DMEM medium (200 μl, approx. 250,000 cells/mil) were added to each well of a D-lysine coated 96-well plate (Corning, Cat No. 3667), and were incubated overnight in a tissue culture incubator at 37° C. under 90% air/10% CO₂. Then, the supplemented DMEM medium was removed with a multichannel pipettor and 200 μl iodine-loading buffer was added to each well. GABA was added to the cells at a final concentration of 100, 30, 10, 3, 1, 0.3, 0.1, or 0 μM with Zymark Rapid plate (Zymark, MA). After the cells were incubated for 5 minutes, they were washed three times with phosphate buffer saline (Invitrogen, CA) and lysed with. 100 μl of cell lysis buffer. The amount of I⁻ in the cell was measured by the SK assay procedure described in Example 1.

Results

FIG. 7 showed that the conductivity of GABAA channel increases with increasing amount of GABA as measured by the SK assay. GABA activated GABAA channel resulting in influx of iodide into the cell. As shown in FIG. 1, the more iodide in the reaction mixture, the less OD405 would be measured from the SK assay. The conductivity of GABAA channel is expressed as the percent activity of the channel, which is defined as: 100*(1−(OD405ₛₐₘₚₗₑ−OD405_{low})/(OD405_{high}−OD405_{low})), wherein OD405ₛₐₘₚₗₑ is measured from the SK assay on cells treated with various concentrations of GABA; OD405_{low} is measured from the SK assay on cells treated with 1000 μM of GABA; and OD405_{high} is measured from the SK assay on cells without GABA treatment. The measured EC₅₀ of GABA, which is the concentration of GABA at which the activity of the GABAA channel is induced by one-half as compared to reactions without GABA, was 294 μM.

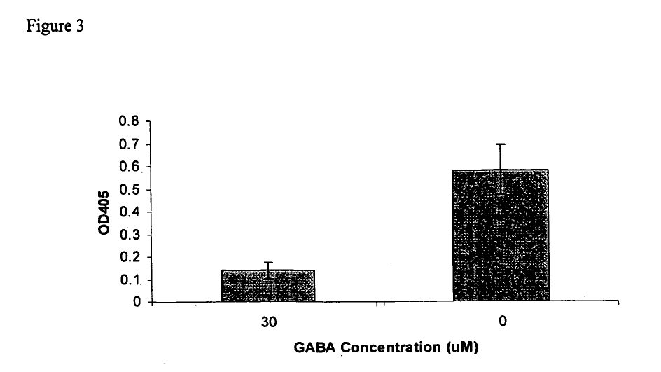

FIG. 3 showed that stimulating cells with 30 μM GABA resulted in approx. 4.5 fold decrease in OD405 from the SK assay as compared to cells not stimulated with GABA. Therefore under the assay condition described herein, a test compound capable of decreasing the conductivity of GABAA could be identified by its ability to cause less than 4.5 fold decrease in OD405 from the SK assay in the presence of 30 μM GABA, as compared to cells not stimulated with GABA.

What is claimed is:

1. A method for measuring chloride channel conductivity, comprising the steps of:
a) contacting a chloride channel with iodide; and
b) calorimetrically detecting the amount of iodide conducted by the chloride channel.

2. The method of claim 1 wherein the method measures the influx of iodide into a cell or membrane vesicle having the chloride channel, wherein the contacting step comprises incubating a cell or membrane vesicle having the chloride channel in a liquid solution comprising iodide and separating the cell or membrane vesicle from the liquid solution; and wherein the calorimetric detection step comprises measuring the amount of iodide inside the cell or membrane vesicle using a colorimetric detection method.

3. The method of claim 2, wherein the cell or membrane vesicle having the chloride channel is obtained from a patient.

4. The method of claim 1 wherein the method measures the efflux of iodide out of an iodide-loaded cell or membrane vesicle having the chloride channel, wherein the contacting step comprises incubating the iodide-loaded cell or membrane vesicle having the chloride channel in a liquid solution that is substantially free of iodine and separating the cell or membrane vesicle from the liquid solution; and wherein the calorimetrically detecting step comprises measuring the amount of iodide inside the cell or membrane vesicle, or in the liquid solution, using a calorimetric detection method.

5. The method of claim 4, wherein the cell or membrane vesicle having the chloride channel is obtained from a patient.

6. The method of claim 4, wherein the colormetricallly detecting step further comprises the step of determining the ratio of the amount of iodide in the solution to the amount of iodide inside the cell or membrane vesicle.

7. The method of claim 1, further comprising the step of opening or activating the chloride channel prior to the step of calorimetrically detecting the amount of iodide conducted by the chloride channel.

8. The method of claim 1, wherein said chloride channel is selected from the group consisting of a voltage-gated chloride channel, a ligand-gated chloride channel, a swelling-activated chloride channel, a calcium-activated chloride channel, and a CLIC chloride channel.

9. The method of claim 8, wherein said chloride channel is a receptor for γ-aminobutyric acid or glycine.

10. The method of claim 8, wherein said chloride channel is cystic fibrosis transmembrane conductance regulator.

11. The method of claim 1, wherein the calorimetric detection method is based on the Sandell and Kolthoff reaction:

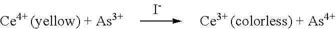

12. The method of claim 1, wherein the calorimetric detection method involves the substrate starch.

13. The method of claim 1, wherein the colorimetric detection method involves the substrate 3,3′,5,5′-tetramethylbenzidine.

14. The method of claim 1, wherein the calorimetric detection method involves the substrate thiocyanate.

15. A method for determining an effect of a test compound on the conductivity of a chloride channel, comprising the steps of
a) contacting the chloride channel with the test compound and iodide;
b) calorimetrically detecting the amount of iodide conducted by the chloride channel; and
c) comparing the amount of iodide detected in step b) with that of a control wherein the chloride channel is not contacted with the test compound.

16. The method of claim 15 wherein the method measures the influx of iodide into a cell or membrane vesicle having the chloride channel, wherein the contacting step first comprises the step of: incubating the cell or membrane vesicle having the chloride channel in a liquid solution containing iodide then contacting the cell or membrane vesicle with the test compound and separating the cell or membrane vesicle from the liquid solution; and, wherein the colorimetrizallly detecting step comprises the step of measuring the amount of iodide inside the cell or membrane vesicle using a colorimetric detection method.

17. The method of claim 15 wherein the method measures the efflux of iodide out of an iodide-loaded cell or iodide loaded membrane vesicle comprising a chloride channel, wherein the contacting step comprises incubating the iodide-loaded cell or membrane vesicle having the chloride channel in a liquid solution that is substantially free of iodine and contacting the cell or membrane vesicle with the test compound and separating the cell or membrane vesicle from the liquid solution; and wherein the detecting step comprises measuring the amount of iodide inside the cell or membrane vesicle, or in the liquid solution, using a colorimetric detection method.

18. The method of claim 17, further comprising a step of determining the ratio of the amount of iodide in the solution to the amount of iodide inside the cell or membrane vesicle.

19. The method of claim 15, further comprising the step of opening or activating the chloride channel prior to the colorimetrizallly detecting step.

20. The method of claim 15, wherein said chloride channel is selected from the group consisting of a voltage-gated chloride channel, a ligand-gated chloride channel, a swelling-activated chloride channel, a calcium-activated chloride channel, and a CLIC chloride channel.

21. The method of claim 20, wherein said chloride channel is a receptor for γ-aminobutyric acid or glycine.

22. The method of claim 20, wherein said chloride channel is cystic fibrosis transmembrane conductance regulator.

23. The method of claim 15, wherein the colorimetric detection method is based on the Sandell and Kolthoff reaction:

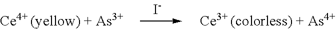

24. The method of claim 15, wherein the calorimetric detection method involves the substrate starch.

25. The method of claim 15, wherein the colorimetric detection method involves the substrate 3,3′,5,5′-tetramethylbenzidine.

26. The method of claim 15, wherein the colorimetric detection method involves the substrate thiocyanate.
